(19) United States
(12) Patent Application Publication
(10) Pub. No.: US 2019/0328772 A1
(43) Pub. Date: Oct. 31, 2019

(54) OPHTHALMIC COMPOSITIONS COMPRISING A CYCLODEXTRIN AS SOLE ACTIVE AGENT

(71) Applicant: WARNEFORD HEALTHCARE LIMITED, Weybridge (GB)

(72) Inventor: Graham Edward PRIESTLEY, Weybridge (GB)

(21) Appl. No.: 16/462,129

(22) PCT Filed: Nov. 17, 2017
(86) PCT No.: PCT/GB2017/000166

§ 371 Date: May 17, 2019

(30) Foreign Application Priority Data
Nov. 18, 2016 (GB) 1619525.7

(51) Int. Cl. A61K 31/724; A61K 9/00; A61P 27/02

(52) U.S. Cl. CPC A61K 31/724; A61P 27/02; A61K 9/0048

(57) ABSTRACT

Pharmaceutical formulations comprising cyclodextrin that are topically applied to the external surface of the human eye, preferably as a liquid. The formulations have been shown to be capable of providing the cyclodextrin to the posterior portions of the eye and are therefore effective in removing drusen and treating other related conditions in the posterior of the eye. Due to the ability to deliver cyclodextrin to the posterior portion of the eye, especially the retina, even though applied topically to the external surface of the eye, the formulations can be used for several related conditions associated with age related degeneration of the human eye, such as wet or dry age related macular degeneration.

This application is a national phase entry under 35 U.S.C. § 371 of PCT International Application Number PCT/GB2017/000166, which claims the benefit of Great Britain patent application number 1619525.7, filed Nov. 18, 2016, each of which is hereby incorporated by reference herein in its entirety.

### FIELD OF THE DISCLOSURE

The disclosure relates to pharmaceutical formulations comprising cyclodextrin that are topically applied to the external surface of the human eye, preferably as a liquid. The formulations have been shown to be capable of providing the cyclodextrin to the posterior portions of the eye and are therefore effective in removing drusen and treating other related conditions in the posterior of the eye. Due to the ability to deliver cyclodextrin to the posterior portion of the eye, especially the retina, even though applied topically to the external surface of the eye, the formulations can be used for several related conditions associated with age related degeneration of the human eye, such as wet or dry age related macular degeneration.

### BACKGROUND

### The Human Eye

The wall surrounding the eye is made up of three distinct layers. The first layer, called the surface layer, is made up of tough collagen. It can be seen in front of the eye as both the sclera and the cornea. The middle layer, called the uveal tract, contains the iris, ciliary body and choroid. The iris is a pigmented segment around the pupil. Essentially a circular muscle fibre, the iris regulates how much light enters the eye. Depending on the brightness, the involuntary muscles relax or stretch allowing more light into the eye when it is dusk or less light when it is bright. The choroid membrane has blood vessels carrying oxygen and other nutrients to the nearby outer portion of the retina. The crystalline lens, located behind the pupil and iris, focuses light rays on the thin, light sensitive retina which is referred to as the third layer. Muscles located in the ciliary body enable the lens to alter its shape for focusing on objects at varying distances. Located within one of the ten layers of the retina are cones and rods, specialized cells that, with the help of visual pigment molecules, enable us to see. Cones are responsible for sharp, discriminating vision and color vision and work best in relatively bright light. About 7 million cones are located within each eye where they are densely packed in the fovea but quickly reduce in numbers toward the periphery of the retina. Rods can function in less light than cones and are mainly used in peripheral vision. About 150 million rods are located within each eye where they are evenly distributed throughout the retina. The cornea consists of three layers, the epithelium which is in contact with the tears, the inner stroma and the endothelium. The lipophilic layered epithelium acts as a barrier to ion transport. Tight junctions located at the epithelium prevent the diffusion of large molecules via the paracellular route but selectively allow some smaller molecules to be absorbed. The stroma is a highly hydrophilic layer and makes up 90% of the cornea. The endothelium maintains corneal hydration. The ciliary epithelium generates aqueous humour generally found between the iris and the cornea providing water-dissolved nutrients to the lens and carrying waste products away from the lens, draining into the Schlemm's canal. The clear and gel-like vitreous humour, located behind the lens, supports and fills the rear two-thirds of the eyeball with a volume of about 4 ml in adults. Made up, almost entirely, of water with glucose, hyaluronic acid, collagen fibres, inorganic salts and ascorbic acid it serves as a pathway for light coming through the lens and maintains the shape of the eyeball.

### Diseases of the Posterior Segment of the Human Eye

The major diseases affecting the posterior part of the human eye are; dry or neovascular age-related macular degeneration [AMD], diabetic retinopathy [DR], diabetic macular oedema [DMO], retinal venous occlusions, proliferative vitreoretinopathy [PVR], inherited retinal diseases, uveitis, and neovascularization or macular oedema due to other conditions. These diseases require attention in order to prevent the loss of vision. Posterior eye diseases present unique anatomical, physiological and biochemical barriers to drug delivery. These result in the failure of conventional dosage forms such as eye drops, ointments and suspensions to deliver any drug to these areas in required concentrations.

Therefore, any prior art disclosure that shows an effect of cyclodextrin in vitro using human eye tissue although of interest does not disclose how cyclodextrin can be reach the posterior segment of the human eye in order to be effective.

Common approaches therefore to the treatment of posterior segment diseases include, but are not limited to, systemic and intravitreal injections and implants into the eye. Systemic drug delivery often results in inadequate retinal concentrations and severe systemic adverse effects. Though intravitreal administration delivers a high concentration of drugs to the retina, the inherent potential side effects like increased intraocular pressure, haemorrhage, cataract formation, and endophthalmitis mean that this is a therapeutic route not without problems. The injection procedure requires attendance at a specialist clinic or hospital to receive treatment. The chronic nature of many retinal diseases requires multiple injections, which are associated with risk of vitreous haemorrhage, retinal detachment, infection and cataract progression as well as a high cost of delivering the therapy. Sustained release implants have overcome some of the disadvantages associated with intravitreal injections; however, other risks may be increased and the surgical procedure and risk of drug precipitation may result in undesirable effects. Recent drugs that are used in sustained release implants for intravitreal use are dexamethasone (Ozurdex) and fluocinolone acetonide (Iluvien). The following products are administered by intravitreal injections as solutions
Bevacizumab (Avastin) 1.25 mg/0.05 ml
Ranibizumab (Lucentis) 0.5 mg/0.05 ml
Triamcinolone acetonide (Kenalog) 0.1 cc of 4 mg/ml (Triesence/Trivaris is alcohol-free preparation that is FDA approved for intraocular use)
Ganciclovir Intravitreal 4 mg/0.1 mL—administer 2 mg in 0.05 mL (twice weekly for CMV Retinitis for 14 days for induction)
Foscarnet Intravitreal 2.4 mg/0.1 ml—administer 1.2 mg in 0.05 mL
Cidofovir—20 micrograms
Fomvirsen—330 micrograms
Methotrexate—400 micrograms
Vancomycin 1 mg/0.1 ml
Ceftazidime 2.25 mg/0.1 ml
Amikacin 0.4 mg/0.1 ml
Amphotericin B; 5 micrograms/0.1 mL
Voriconazole—50-100 micrograms/0.1 mL
Dexamethasone 0.4 mg/0.1 ml

The volume that can be safely injected into the eye is limited. Typically, the maximum amount considered feasible is up to 0.1 ml when a liquid. This places a constraint on the amount of drug injected depending upon solubility of the drug in such a small volume of water. Similarly, the volume of any implant is constrained. These routes may also need theatre time, trained staff and have inherent risk as discussed above.

### Diseases of the Human Retina

Age-related macular degeneration (AMD) is the leading cause of blindness in the elderly, with an incidence of about 20% in adults 65 years of age increasing to 37% in individuals 75 years or older. Non-exudative (dry) AMD is characterized by drusen accumulation and atrophy of rod and cone photoreceptors in the outer retina, retinal pigment epithelium (RPE), Bruch's membrane and choriocapillaries. Neovascular (wet) AMD occurs because of choroidal neovascularization. The pathogenesis of retinal degenerative diseases, such as AMD, is multifaceted and can be triggered by environmental factors in normal individuals or in those who are genetically predisposed. To date more than 100 genes have been mapped or cloned that may be associated with various outer retinal degenerations.

Early stages of macular degeneration have been shown to be slowed in some eyes with combinations of antioxidants or anti-inflammatory agents. There is the AREDS formulation (Age Related Disease Study) where researchers use oral supplementation of 500 milligrams of vitamin C; 400 International Units of vitamin E; 15 milligrams of beta-carotene (often labelled as equivalent to 25,000 International Units of vitamin A); 80 milligrams of zinc as zinc oxide; and two milligrams of copper as cupric oxide. Copper was added to the AREDS formulations containing zinc to prevent copper deficiency anaemia, a condition associated with high levels of zinc intake. AREDS was found to have efficacy is slowing AMD. It reduced the rate of advanced AMD in people at high risk by about 25 percent over a 6-year period. A new formulation AREDS2 formulation which are oral supplementation of high doses of macular xanthophylls (lutein and zeaxanthin) and/or omega-3 LCPUFAs (DHA and EPA) for the treatment of AMD and cataract—see www.areds2.org. was also investigated In the AREDS2 trial, adding DHA/EPA or lutein/zeaxanthin to the original AREDS formulation (containing beta-carotene) had no additional overall effect on the risk of advanced AMD. However, trial participants who took AREDS containing lutein/zeaxanthin and no beta-carotene had a slight reduction in the risk of advanced AMD, compared to those who took AREDS with beta-carotene. Also, for a subgroup of participants with very low levels of lutein/zeaxanthin in their diet, adding these supplements to the AREDS formulation helped lower their risk of advanced AMD. Finally, former smokers who took AREDS with beta-carotene had a higher incidence of lung cancer. The investigators found no significant changes in the effectiveness of the formulation when they removed beta-carotene or lowered zinc.

Advanced stages of macular degeneration that lead to severe vision loss can be treated either by surgical removal of membranes from the subretinal space, laser photocoagulation, photodynamic therapy, or, more commonly now, with VEGF blockers (aflibercept, ranibizumab and bevacizumab) in patients with exudative AMD. No approved treatments are currently approved for the advanced form of dry AMD, also known as Geographic Atrophy. Laser treatment is also used in the treatment of diabetic retinopathy. It is important to note that both laser photocoagulation of the retina and surgical excision of subretinal membranes or intravitreal membranes results in the destruction of viable retinal neurons.

There is currently no approved topical treatment for the treatment/prevention of AMD or any disease of the posterior segment of the human eye.

Drusen are clinically visible collections of lipids and proteins plus other waste materials within the retina. They are an early sign of macular degeneration and have a prognostic value. The hallmark of the disease, caused by the accumulation of extracellular material (drusen) upon, within, or between the retinal pigment epithelium and Bruch's membrane. Studies have shown that drusen and reticular pseudodrusen form as extracellular deposits between the retinal pigment epithelium (RPE) basal lamina and the inner collagenous layer of Bruch's membrane. They cause stretching of the RPE monolayer and physical displacement of the RPE from its immediate vascular supply, the choriocapillaries. This displacement likely creates a physical barrier that may impede normal metabolite and waste diffusion between the choriocapillaries and the neural retina. In this paradigm, cellular waste products may be concentrated near the RPE and the diffusion of oxygen, glucose, and other nutritive or regulatory serum-associated molecules required to maintain the health of the outer retina and RPE are inhibited. Thus, leading to a continuing build-up of waste material. It has also been suggested that drusen perturb photoreceptor cell function by placing pressure on rods and cones and/or by distorting photoreceptor cell alignment.

Drusen deposits have been shown to be a significant risk factor for the development of dry AMD. In fact, the underlying cause of dry AMD is thought to be related to drusen deposits. Drusen deposits are known to be composed of proteins and lipids (i.e., phospholipids, neutral lipids, cerebrosides, gangliosides, numerous other proteins, etc.) that are derived from systemic, choriocapillaries and retinal sources. There is currently no approved treatment for halting or reversing loss of vision resulting from dry AMD or geographic atrophy by any means of delivery. There is no current available treatment for the removal of drusen, or reduction in the size and/or quality of drusen, or the prevention of their build up.

There are different kinds of drusen. Reticular Pseudodrusen. “Hard” drusen are small, distinct and far away from one another. This type of drusen may not cause vision problems for a long time, if at all. “Soft” drusen are large and cluster closer together. Their edges are not as clearly defined as hard drusen. This soft type of drusen increases the risk for AMD.

Drusen and drusen like material also deposit in the retina-choroid interface in other conditions such as Sorsby's Fundus Dystrophy and Malattia Leventinese.

Drusen occur naturally with age. They are believed to be the result of the eye's failure to eliminate waste products produced in the cells of the eye. Drusen also represent the clinically visible feature of a thickened Bruch's membrane, a membrane that separates the retina from the choroid. The Bruch's membrane becomes lipid laden with age and prevents the exchange of nutrients and removal of waste material across the retina-choroid interface. Removing the lipids from the Bruch's membrane or reducing the thickness of this membrane is key to improving conductivity between retina and choroid. Laser treatment has been attempted to improve the flow through the Bruch's membrane, but the results were unsatisfactory. Therefore, agents that can prevent or delay the ageing changes of the Bruch's membrane are key to reducing the development of age related changes and AMD.

Separately it has also been found that lipids also accumulate in the photoreceptor membranes with age and this may also contribute to the visual functional losses that is experienced in ageing. Currently, there are no treatment options for clearing excessive lipid accumulation from the retina and choroid that can potentially prevent ageing changes manifested clinically as drusen.

The exact relationship between degenerative macular disease and drusen is not clear. Scientists are uncertain whether drusen cause AMD or whether AMD and drusen are caused by the same process but are otherwise unrelated. The presence of soft drusen is a sign of AMD. However, recently it has been found that small reductions in drusen caused by light therapy have led to an improvement in vision (Photobiomodulation induces drusen regression with improvements in visual acuity and contrast sensitivity in subjects with dry AMD Merry et al ARVO 2016 Abstract 4439) thus suggesting for the first time that by reducing the size or even eliminating drusen from the retina then AMD progression could be halted and possibly even reversed. To establish the effectiveness of treatment it is necessary to show an improvement in or at least a stabilization of visual acuity and anatomic evidence of slowing or halting degeneration, which often takes one to two years to manifest meaningfully. However, the visual reduction in the number and size of drusen in a patient would be a key indicator of the success of a medicine in treating macular degeneration.

Therefore, the elimination or prevention of drusen (as a hallmark of ageing and AMD) is thought to be a critical factor in treating or preventing AMD.

### Topical Delivery to the Posterior Segment of the Human Eye

A non-invasive topical drug delivery systems in the form of eye drops applied to external surface of the human eye would circumvent most of the treatment problems mentioned above associated with intra vitreal administration and would greatly reduce the cost and burden of therapy. Administration of drugs in the form of eye drops for diseases of the posterior segment of the human eye has several advantages; it allows self-administration, localized therapeutic effect, a non-invasive and painless mode of drug administration, and high patient compliance. It also eliminates the risk of the invasive procedures.

However, the following significant challenges exist that prevent such an option being successful;
1. The normal mean tear volume is 6.5 μl. The volume of liquid that remains in the eye is very low. Above this amount the fluid drains into the nose where certain drugs can enter the blood stream and circulate systemically. Topical application in the form of eye drops is the most common method used to treat both the outside of the eye, such as dry eyes, and to provide intraocular treatment (of the anterior segment of the eye) with absorption through the cornea, such as glaucoma using a range of drugs (such as latanoprost, travoprost, brinzolamide and dorzolamide). Drugs are topically applied as aqueous solutions (latanoprost, travoprost and dorzolamide) or as aqueous suspensions (brinzolamide) that are pharmacologically potent at very low concentrations.
2. Short contact time of the drug (1-2 min) on the surface of the eye. Washing away by tear fluid followed by drainage into the nasolacrimal leads to a rapid elimination of the topically applied drug. The three-layered cornea also limits the absorption with the epithelium limiting the absorption of hydrophilic drugs and the stroma limiting the absorption of lipophilic drugs. Mucins secreted to protect the ocular surface also forms a hydrophilic layer over the tears.
3. Conjunctival drug absorption into the eye is limited. Blood-retinal barriers and blood-aqueous barriers express tight junctions which limit drug penetration from the systemic bloodstream into the intraocular environment. Systemic administration of drugs will thus, in most cases, not be able to reach therapeutic levels in the eye and orally administered drugs will not reach therapeutic levels in the eye unless given in very high dose. These high doses could result in systemic side effects

The current understanding is that the penetration of drugs into the cornea and conjunctiva is driven by; the concentration gradient, lipophilicity and molecular weight of the drug. The epithelial layers of the cornea and conjunctiva act as rate-limiting barriers for drug absorption. Depending on the lipophilicity, the drug will enter the conjunctival or corneal epithelium through the paracellular and transcellular routes. The hydrophilic drugs such as atenolol and inulin enter the epithelial layers via the paracellular route, while lipophilic drugs such as timolol and propranolol enters through the transcellular pathway. The intercellular space of corneal and conjunctival epithelia is sealed by the junctional complexes that hinder the transport of hydrophilic compounds. The rate of paracellular penetration decreases with an increase in molecular size. The conjunctiva is 15-25 times more permeable to hydrophilic compounds compared to the cornea. This is mainly because of the larger paracellular pore diameter of the conjunctiva (3.0 nm±1.6), which allows the permeation of molecules with a size ranging between 5-10 kDa. The paracellular pore diameter of the corneal epithelium is 2.0 nm±0.2, and hence it allows the paracellular permeation of molecules with size<500 Da.

Various means are known to help the penetration of drugs through the tight paracellular junctions and can be enhanced by the addition of chelating agents such as EDTA and permeation enhancers such as polyoxyethylene-20-stearyl ether. Drugs entering through the conjunctiva route can be rapidly cleared due to the presence of blood and lymphatic circulation. A fraction of the drug escaping conjunctival barriers will permeate through the sclera, which is then challenged by the choroidal circulation and the retinal pigment epithelium, a monolayer of cells with tight junctions (outer blood-retinal barrier), before reaching neural retina. Therefore, the route through the cornea is not seen as viable for the delivery of drugs for treating the posterior section of the eye—especially large molecules that cannot penetrate the cornea.

### Cyclodextrins

Cyclodextrins (CD) are manufactured by bacterial fermentation of starch followed by product purification. First believed to be discovered in 1891 by a French scientist named A. Villiers, the different CDs were not isolated until years later. The isolation step was tiresome which resulted in high prices. With the biotechnological advances in the early 1970s came new ways to produce CD and high-grade CDs were available at affordable prices.

The molecular weight average (Mw) of the simplest cyclodextrin is 980 and typical cyclodextrin polymers are >1000 Da to around 1300 Da.

Cyclodextrins have long been known to be solubilising agents through their ability to complex with lipophilic materials to form inclusion complexes (clathrates). They are also known to improve the stability of numerous compounds. They are also known to bind molecules through their external hydrophilic surface. The cyclic nature of the polysaccharide forms a pocket into which the compound can fit. Different substituents on the polysaccharide can lead to the ability to bind different compounds. Generally, they are believed to be safe and are accepted as excipients that are injected directly into patients or can be ingested orally in relatively large amounts with no adverse effects.

Cyclodextrins have been proposed to form inclusion complexes with corticosteroids. Corticosteroids are commonly used in many eye conditions. Examples are dexamethasone which is commonly used topically on the corneal for treating corneal inflammation. Also, dexamethasone and triamcinolone and fluocinolone are used as intravitreal injections for treating various forms of macular oedema—dexamethasone and fluocinolone are inserted as a slow release solid formulation. It is well known that these drugs do not easily pass through the cornea due to poor aqueous solubility and poor dissolution. It has been found that when the drugs are used with cyclodextrins the inclusion complex somehow helps drugs to pass through the cornea. It is well known that the cyclodextrin cannot pass through the cornea. The mechanism by which dexamethasone is delivered to the posterior part of the eye using cyclodextrins is explained later below.

Cyclodextrins have been increasingly used in pharmaceutical formulations, in low concentrations, as a solubilizing or penetrating agent. For example, U.S. Pat. Nos. 5,919,813 and 6,028,099 are directed to the treatment of diabetic retinopathy or choroidal neovascularization via administration of protein tyrosine kinase inhibitors, such as genistein or derivatives thereof. In describing the pharmaceutical compositions for use in the claimed treatment methods, the applicants state that the PTK inhibitor can be formulated as cyclodextrin inclusion complexes, among other types of suggested excipients for pharmaceutical formulations. These patents do not suggest the use of cyclodextrins in therapeutically effective amounts as active ingredients for the solubilisation of drusen or the treatment of dry AMD.

Cyclodextrins have been utilized as a solubilising agent or penetration-enhancing agent in pharmaceutical formulations for several years. For example, U.S. Pat. Nos. 4,978,532; 5,120,546; 5,288,497; and 5,288,498 discuss the use of cyclodextrins as a solubilizing agent or penetration-enhancing agent. In particular, cyclodextrins have been used to form an inclusion complex with dexamethasone and these have been used to deliver significantly higher amounts of dexamethasone to the posterior segment of the eye when applied topically. However, the increased delivery of dexamethasone is achieved by improving the permeation of dexamethasone through the cornea.

Corticosteroids are generally lipophilic and very poorly soluble in water. By forming an inclusion complex with cyclodextrins the hydrophilic cyclodextrin can transport the lyophilic corticosteroid through the lacrimal aqueous environment to the surface of the cornea and from there the corticosteroid is released and passes through into the lipophilic cornea environment. The cyclodextrins helps drive the corticosteroid through by increasing the surface concentration of dexamethasone available at the cornea and improving natural diffusion processes out of the inclusion complex and into the more lipophilic environment of the cornea (see Acta. Ophthalmol. Scan 2002: 80 144-150 & Acta Ophthamol. Scand. 2007:85 598-602). Also, it has been shown that a significant amount of dexamethasone, when applied topically, with or without cyclodextrin reaches the posterior segment of the rabbit eye through a systemic route (the drug being absorbed from the eyedrop into the blood stream—see Acta Ophthamol. Scand. 2007:85 598-602). The product is being developed as a nanoparticulate (NP) suspension (not an aqueous solution) form of cyclodextrin with dexamethasone—called DexNP. The NP cyclodextrin does not deliver all drugs. It was found to not work for dorzolamide and no enhanced amount of dorzolamide was delivered to the posterior part of the eye (see Acta Ophthamol 2014; 92 550-556).

Additionally, cyclodextrins have been studied as agents for use in artificial tears such as in combination with cholesterol—the amount of cyclodextrin being in an amount of 20%—for use in treating mild dry eye syndrome (see Proceedings of the International Symposium on Cyclodextrins. Vol 8; 1996, 391-394).

U.S. Pat. No. 8,158,609 (Novartis) discloses the use of cyclodextrins for treating drusen. However, the experiment in the patent only shows that a human eye suffering from AMD, which is taken from a dead patient, can have the drusen removed when it is cut up into sections and then immersed in solutions of cyclodextrin for 30 mins, 1 hr, 2 hrs, 4 hrs and 15 hrs at room temperature and finally only at 24 hours is it shown to be effective. In this first experiment it is reported that “Drusen shows no change after treating the section with 40 ml of different concentrations selected from hydroxypropyl β-cyclodextrin at 37° C. for 24 hours by H&E staining which indicated that hydroxypropyl β-cyclodextrin is not able to dissolve the solid drusen in this experimental condition” It was subsequently found that at concentrations of 25-40% at 24 hours it was able to partially remove mouse brown fat tissue—used as a positive control. Subsequently an experiment was performed with 40 ml of 25% hydroxypropyl β-cyclodextrin at 37° C. for 24 hours that showed that there was “efficacy in dissolving the lipid component in drusen.” in sections of a cut up human eye. The claims as granted in U.S. Pat. No. 8,158,609 are limited to intravitreal injection of cyclodextrin. In the submission of the patentee on the 20 Dec. 2006 the patentee states that cyclodextrins cannot penetrate the eye via the topical route to distinguish over the disclosure of several references showing topical application of cyclodextrins. By making such an admission the claims had to be limited to only intravitreal administration into the eye by the US applicant. It is not surprising that cyclodextrin is capable of removing fatty deposits given its known abilities to form complexes with many different molecules. The question really is how a cyclodextrin can be delivered to the posterior segment of the human eye of a patient?

It is not explained in U.S. Pat. No. 8,158,609 how any amount of cyclodextrin can get access into the eye except by intravitreal injection. It is completely impractical to use intravitreal injection based upon the experiments and the science is speculative. The experiment uses 25% concentration of cyclodextrin in 40 ml of water to show efficacy and at 24 hours. It is not possible to administer 10 g of cyclodextrin into the eye by intravitreal injection—even assuming no volume of a diluent. The patent attorney drafting the patent suggest that the composition can be applied to the eye as a topical therapy but provides no proof or evidence that it can reach the posterior part of the eye. At the time of the patent and up to filing this patent no one believed that a molecule of the size of cyclodextrin could enter the eye. To do so the compound would have to pass through the conjunctiva and cornea. The molecule is too large.

WO2012100142—Cornell University—discloses that cyclodextrins are capable of binding lipofuscin bisretenoids (A2E) found in the retinal pigment epithelial cells (RPE). The patentee demonstrates through in vitro work which cyclodextrins are best at binding A2E by simply mixing solutions of A2E with cyclodextrins and measuring a spectral shift caused by binding. Subsequent experiments use a RPE cell line grown with A2E containing media. The cells are incubated for three days in media containing 1 μM of a cyclodextrin. In further experiments sections of mouse eye comprising the RPE and the choriocapillaries and the sclera were bathed in media containing 2 μM cyclodextrin for 36 hours. In example 9 mouse eyes receive a sub-tenon injection of 4000 mg/kg of cyclodextrin and then the eyes are dissected and inspected after 48 h. The patent filing focusses all of its disclosure on retinal lipofuscin damage to the RPE. It offers no suggestion on how cyclodextrins can be administered to the surface of the eye and reach the retinal layers and affect drusen.

WO2016168772—Sens Research Foundation—discloses that cyclodextrins are effective in slowing down ageing processes. They focus on its ability to bind lipofuscin and in particular non-bisretenoid lipofuscin, i.e. non retinal lipofuscin. Such products are directed to topical treatment on the skin. The data Example 1 is looking at skin cells. Additional studies are looking at effects on fibroblast cells (associated with collagen production). The work concludes that cyclodextrins have their effects in lowering lipofuscin from cells by the cyclodextrin manipulating the total cellular cholesterol as well as the cholesterol content of the lysosomal membrane.

US2016/0151410—Columbia University—discloses a method of treating a patient with wet AMD by administrating to a patient an amount of a modified alpha-cyclodextrin. The modified alpha cyclodextrin binds to bioactive lipids that are characterised as having a single chain of fatty acid, such as lysophospholipids. The principle mechanism for its action is postulated that by removing these compounds then there is mechanistic change in complement activation, RPE cell death and neovascularisation. In particular they associate the action of cyclodextrins being beneficial in patients of certain genotypes where expression of lysophosphatidtylcholine (LIPC) and that LIPC leads to the a number of pathways and generation of bioactive lysophospholipids and that reduction of these will lead to improved outcomes in wet AMD. However, no information is provided how the cyclodextrin can be provided to the retinal areas of the eye from a topical application to the external surface of the eye.

### SUMMARY

We have found that when a cyclodextrin composition is applied as a topical therapy it can enter the eye and it is effective in removing drusen—even at greatly reduced concentrations than those suggested by U.S. Pat. No. 8,158,609 or other prior art references. Whilst not wishing to be bound by this statement we believe that it is still impossible for the cyclodextrin to enter the eye through the cornea and we believe that the cyclodextrin enters the posterior segment of the human the eye via the ciliary body and from there into the far peripheral retina and then moves centrally along the retina. Regardless of the mechanism we have found that the application of a topical solution of cyclodextrin to patients does shrink the size and extent of drusen. That the amount of cyclodextrin needed is surprisingly low when applied topically.

Therefore, we present as a feature of the invention:

An ophthalmic topical composition comprising a cyclodextrin and any pharmaceutically acceptable excipient therefor.

The composition is alternatively to comprising; containing, consisting of or consisting essentially of a cyclodextrin. The composition is for application to the external surface of the human eye. The composition is for use in the treatment of prevention of diseases of the posterior segment of the human eye as set out herein. Also disclosed are methods of treatment (that include prevention) of one or more diseases of the posterior segment of the eye, as set out herein, comprising applying a composition of the invention onto the external surface of the eye of a patient in need of such treatment in a pharmaceutically efficacious amount.

Preferred features are those wherein, the cyclodextrin is the sole active ingredient. The topical composition may be a pharmaceutical formulation.

Cyclodextrins (CD) are cyclic oligosaccharides consisting of six (α-CD), seven (β-CD), eight (γ-CD) or more D-glucopyranose units linked with α-(1,4) bonds. Due to chair structure of the glucopyranose units the CD molecule is shaped like a truncated cone with the primary hydroxyl (—OH) groups extending from the narrow edge and the secondary hydroxyl groups from the wider edge. The hydroxyl groups extending from the edges of the molecule give the CD a hydrophilic outer surface while the inner cavity, lined with carbons and ethereal oxygen of the glucose residue, is rather lipophilic. However, due to high crystal lattice energy and intra-molecular hydrogen bonding between C-2 and C-3 hydroxyl groups, the aqueous solubility of parent CDs and their complexes is limited, especially for βCD. The low aqueous solubility has been overcome by creating CD derivatives, which are of pharmaceutical interest and include hydroxypropylated-βCD and -γCD (HPβCD and HPγCD), randomly methylated-βCD (RMβCD) and sulfobutyl ether βCD sodium salt (SBEβCD). The physicochemical properties of the derivatives depend on the structure, location and number of the substituents. The CD derivatives also have different hydrophobic cavity volume compared to the parent molecules. A CD may exist as a salt form and pharmaceutically acceptable salt forms of cyclodextrins are within the term “cyclodextrin”.

Natural CDs are more resistant towards starch hydrolysing enzymes and non-enzymatic hydrolysis than the linear oligosaccharides. In aqueous solutions, non-enzymatic hydrolysis of the α-acetal linkages produces glucose maltose and linear oligosaccharides. The derivatives are degraded at similar speed with ring opening the dominant pathway.

The cyclodextrin is preferably an α, β or γcyclodextrin and is preferably a βcyclodextrin. Preferably the βcyclodextrin is selected from; β-cyclodextrin sulfobutyl ether and salts thereof and hydroxypropyl-β-cyclodextrin and salts thereof. Preferably the grade of cyclodextrin used is injectable grade.

β-Cyclodextrin sulfobutyl ether is a substance in which hydroxyl groups at the 2-, 3- and 6-positions in glucopyranose constituting β-cyclodextrin have been substituted suitably with sulfobutyloxy groups, and the salts of this substance are typically those in which a hydroxysulfonyl group (HOSO2-) of the substance forms a salt with an alkali metal such as sodium or an alkaline earth metal such as calcium.

β-Cyclodextrin is a substance having 7 glucopyranoses bound in a cyclic form via alpha-1,4-linkages, and thus one molecule of beta-cyclodextrin has 21 hydroxyl groups in total derived from the 2-, 3- and 6-positions in the glucopyranoses constituting beta-cyclodextrin, among which preferably about 7 hydroxyl groups have been substituted with sulfobutyloxy groups and simultaneously the hydroxysulfonyl group (HOSO2-) has formed preferably a sodium salt, and such substance includes, for example CAPTISOL.

Hydroxypropyl-β-cyclodextrin refers to a substance in which the 21 hydroxyl groups in beta-cyclodextrin have been substituted with 2-hydroxypropyloxy groups, and usually this substance is preferably the one in which out of the 21 hydroxyl groups from 4 to 8 hydroxyl groups have been substituted with 2-hydroxypropyloxy, and particularly preferably having a degree of substitution of 4 to 5, and such substance include e.g. Celdex HP-β-CD with a degree of substitution of 4.6-7.6. It is important to appreciate that the degree of substitution is an average and numbers between whole integers is possible. A preferred commercially available product is Kleptose HPB (preferably injectable grade and preferably pyrogen free), which has a Mw of 1380-1480 Daltons with an average substation of 4.5 (with a range of substitution from 1 to 10).

Preferably the cyclodextrin is sulfobutylether of 13-CD (SBE-β-CD), the hydroxypropyl derivative of 13-CD (HP-β-CD), and the randomly methylated β-CD (RM-β-CD). The composition may contain one, two or three of these CDs described. Preferably when used as a mixture the mixture contains only sulfobutylether of β-CD (SBE-β-CD) and hydroxypropyl derivative of β-CD (HP-β-CD) is a ratio of 25:75 to 75:25.

Preferably the cyclodextrin is administered as a sterile aqueous solution. the total concentration of cyclodextrin is less than 25% wt., ideally less than 24, 23, 22, 21, 20, 19, 18, 17, 16, 15, 14, 13, 12, 11, 10, 9, 8, 7, 6 and 5% wt. Preferably the concentration of cyclodextrin is greater than 1, 2, 3, 4, 5, 6, 7, 8, 9, 10, 11, 12, 13, 14, 15, 16, 17, 18, 19, 20, 21 and 22% wt. What is suprising is that given the barriers presented to ocular penetration of CDs to the posterior segment of the human eye that concentrations so low can be effective. Whilst not wishing to be bound by this statement it is thought that each cyclodextrin molecule that reaches the posterior section of the human eye will have a direct effect in sequestering waste material and that these effects are cumulative. Therefore, the importance is the realisation that the CD will reach the posterior segment of the human eye, when topically applied, and that the continuing treatment over an extended period of time will have an effect in ameliorating the conditions described herein even when concentrations of the CD applied are relatively low.

Toxicological studies have demonstrated that orally administered CDs are safe since they are unable to permeate lipophilic membranes such as gastrointestinal mucosa and skin, apart from RMβCD which has higher bioavailability due to increased lipophilicity. Nevertheless, after a complex formation between the CD and the guest molecule, their ability to interact with biological membranes is greatly reduced and usually only seen in vivo at relatively high concentrations.

Aqueous eye drops containing large proportion of CD (12-25%) along with cholesterol given to dry eye patient results in the formation of crust in the eyelids with consequent irritation Stefánsson E., Thórisdóttir S., Gu [symbol] mundsson Ó. G., Loftsson T., Fri [symbol] riksdóttir H., Kristinsson J. K. (1996) 2-Hydroxypropyl-β-Cyclodextrin in Eye Drops. Evaluation of Artificial Tear-Drops In Human Patients. In: Szejtli J., Szente L. (eds) Proceedings of the Eighth International Symposium on Cyclodextrins. Springer, Dordrecht

The EMEA has issued draft guidance [20 Nov. 2014 EMA/CHMP/333892/2013 Committee for Human Medicinal Products (CHMP) Background review for cyclodextrins used as excipients In the context of the revision of the guideline on ‘Excipients in the label and package leaflet of medicinal products for human use’ (CPMP/463/00 Rev. 1)] on the use of cyclodextrins in pharmaceutical products and in its conclusion on page 8 it states that

“Conclusion Cyclodextrins enhance drug penetration into the eye. Concentrations of 4% α-CD and 5% RM-β-CD can be toxic to the corneal epithelium of rabbits. Solutions of 10% SBE-β-CD and 12.5% HP-β-CD are found not to be toxic or irritating in rabbit eyes.”

The permitted daily exposure parentally as issued by the EMEA in their guidance note is

“Parenteral α-CD (0.2 mg/kg/day) γ-CD (0.8 mg/kg/day) HP-β-CD (0.2 mg/kg/day) HP-β-CD (320 mg/kg/day) SBE-β-CD (0.32 mg/kg/day) SBE-β-CD PDE (280 mg/kg/day)”

We have found that the cyclodextrin when applied topically to the exterior surface of the human eye does enter the posterior segment of the eye and in particular it can enter the retina, the choroid or the Bruch's membrane we mean >45, 50, 55, 50, 65, 70, 75, 80, 85, 90, 95, 97, 98, 99 and 100%). We have also found that not only does the cyclodextrin enter the eye to the posterior segment of the human eye but that also the cyclodextrin that enters the eye also quickly leaves the eye within less than ten hours (less than 9, 8, 7, 6, 5, 4, 3, 2, 1 hour), we presume by the capillaris and then into the systemic vasculature. This is important since the cyclodextrin carries with it the waste material that it has sequestered and there is no possibility of the cyclodextrin accumulating in the eye or re-deplying that waster material into another part of the eye.

This means that the composition can be used multiple times a day up to the daily amount of cyclodextrin allowed to be injected (the cyclodextrin after having entered and exited the eye enters the systemic system). It also means that it is safe to use over extended periods of time.

The water used is preferably water for injection grade.

An ophthalmic composition where in the remaining part of the composition that is not cyclodextrin or water is selected from one of more of the following excipients;
carriers,
solubilisers,
stabilisers
buffering agent
pH adjusting agent,
tonicity agent,
wetting agent,
viscosity enhancing agent, and
preservative.

Other customary ophthalmically-acceptable excipients and additives known to the person skilled in the art may be comprised in an above composition, for example those of the type mentioned below, especially carriers, stabilizers, solubilisers, tonicity enhancing agents, buffer substances, preservatives, viscosity enhancing agent, and other excipients. Such compositions are prepared in a manner known, for example by mixing the active ingredients with the corresponding excipients and/or additives to form corresponding ophthalmic compositions.

Carriers used in accordance to the present invention are preferably suitable for topical administration, and are apart from water, mixtures of water and water-miscible solvents, such as Cₗ- to C7-alkanols, vegetable oils or mineral oils comprising from 0.5 to 5% by weight hydroxyethylcellulose, ethyl oleate, carboxymethyl-cellulose, polyvinyl-pyrrolidone and other non-toxic water-soluble polymers for ophthalmic uses, such as, for example, cellulose derivatives, such as methylcellulose, alkali metal salts of carboxy-methylcellulose, hydroxymethylcellulose, hydroxyethylcellulose, methylhydroxypropyl-cellulose and hydroxypropylcellulose, acrylates or methacrylates, such as salts of polyacrylic acid or ethyl acrylate, polyacrylamides, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. Preferred carriers are water, cellulose derivatives, such as methylcellulose, alkali metal salts of carboxymethylcellulose, hydroxymethylcellulose, hydroxyethylcellulose, methylhydroxypropylcellulose and hydroxypropylcellulose, neutral Carbopol, or mixtures thereof.

The solubilisers used for an ophthalmic composition of the present invention are, for example, tyloxapol, fatty acid glycerol polyethylene glycol esters, fatty acid polyethylene glycol esters, polyethylene glycols, glycerol ethers or mixtures of those compounds. A specific example of an especially preferred solubilisers is a reaction product of castor oil and ethylene oxide, for example the commercial products Cremophor EL® or Cremophor RH 40*. Reaction products of castor oil and ethylene oxide have proven to be particularly good Solubilisers that are tolerated extremely well by the eye. Another preferred solubilise is tyloxapol.

Examples of buffer substances are acetate, ascorbate, borate, hydrogen carbonate/carbonate, citrate, gluconate, lactate, phosphate, propionate and TRIS (tromethamine) buffers. Tromethamine and borate buffer are preferred buffers. The amount of buffer substance added is, for example, that necessary to ensure and maintain a physiologically tolerable pH range.

Examples of preservatives are quaternary ammonium salts such as benzalkonium chloride, benzoxonium chloride or polyquats (polymeric quaternary ammonium salts, being specifically disclosed in the Canadian Patent No. 1069522), alkyl-mercury salts of thiosalicylic acid, such as, for example, thiomersal, phenylmercuric nitrate, phenylmercuric acetate or phenylmercuric borate, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, or sorbic acid. Preferred preservatives are quaternary ammonium salts, alkyl-mercury salts and parabens. Where appropriate, a sufficient amount of preservative is added to the ophthalmic composition to ensure protection against secondary contaminations during use caused by bacteria and fungi.

### pH, Osmolality, Surface Tension

The pH range is typically in the range of from 5 to 9, preferably from 5.2 to 8.5 and more preferably from 5.5 to 8.2. Ideally, ophthalmic solutions should have the same pH as the lacrimal fluid (7.4), but pH values from 7 to 9 are tolerated by the eye without marked irritation. The buffer capacity of the lacrimal fluid (0.01 ml) should not be exceeded due to increased tear production and eye movement, resulting in increased eye drop clearance.

The lacrimal fluid is isotonic (i.e. has the same tonicity) with blood with 287 mOsm/l. Ideally, an ophthalmic solution should have the same tonicity values as the lacrimal fluid but the eye can tolerate a rather broad range of tonicity from ˜205-683 mOsm/l (USP, 1995). Preferably the osmolality is 300-500 mOsm/l. The tonicity is adjusted by the use of suitable agents as disclosed herein. The osmolality in normal tear fluid is from 310 to 340 mOsmol/Kg and ideally the formulation has an osmolality that is inside this range and the amounts of the tonicity ingredients are adjusted accordingly. In certain cases, the osmolality might be desired to be higher (hyper osmolality formulation) or lower (hypo osmolality formulation) and this depends upon other factors such as pH and colloidal osmolality effects caused by any viscosity agent added into the formulation. The osmolality may be measured using a freezing-point depression osmometer, following the procedure set out in the US Pharmacopeia, USP 34, National Formulary 29, 2011, chapter 785. Tonicity enhancing agents are, for example, ionic compounds, such as alkali metal or alkaline earth metal halides, such as, for example, CaCl2), KBr, KCl, LiCl, NaI, NaBr or NaCl, or boric acid. Non-ionic tonicity enhancing agents are, for example, urea, glycerol, sorbitol, mannitol, propylene glycol, or dextrose. For example, sufficient tonicity enhancing agent is added to impart to the ready-for-use ophthalmic composition an osmolality of approximately from 50 to 1000 mOsmol/kg, preferred from 100 to 400 mOsmol/kg, more preferred from 200 to 400 mOsmol/kg and even more preferred from 250 to 350 mOsmol/kg, ideally 280-300 mOsmol/kg.

The surface tension of the lacrimal fluid ranges from 40 to 50 mN/m. Low surface tension provides good spreading effect on the cornea possibly improving the contact between the drug and corneal epithelium. Preferably the surface tension is from 20 to 60 mN/m—The surface tension is adjusted using wetting agents as disclosed herein.

An ophthalmic composition as described herein wherein, the composition comprises a component that acts as an artificial tear and/or an eye lubricant.

We describe a method of treating a disease of the posterior segment of the human eye by the topical application to the exterior surface of the eye a pharmaceutically effective amount of a composition comprising a cyclodextrin.

An ophthalmic topical composition as described herein wherein, the composition is for use in the removal of drusen from the eye of a human patient. An ophthalmic topical composition as described herein for removal and/or reduction in the size of drusen and lipid accumulation upon within or between the retinal pigment epithelium and Bruch's membrane of the human eye.

An ophthalmic topical composition as described herein for removal of drusen and/or the reduction in the size of the drusen in the macula. An ophthalmic topical composition as described herein for treating or preventing Sorsby's Fundus Dystrophy and Malattia Leventinese. An ophthalmic topical composition as described herein for the removal of cellular waste products produced in the cells of the eye, especially from the posterior section of the eye, especially the retina, and especially the Bruch's membrane of the retina. An ophthalmic topical composition as described herein for the removal of lipids from the Bruch's membrane or reducing the thickness of the Bruch's membrane. An ophthalmic topical composition as described herein for the removal of lipid accumulation in the photoreceptor membranes. An ophthalmic topical composition as described herein for prevention of lipid accumulation in the photoreceptor membranes. An ophthalmic topical composition as described herein wherein, the composition is for use in the prevention of the formation of drusen in the eye of a human patient. An ophthalmic topical composition as described herein wherein the patient has previously been identified as being susceptible to the development of dry or wet age related macular degeneration. An ophthalmic composition as described herein for preventing dry or wet age related macular degeneration. An ophthalmic composition as described herein for treating dry or wet age related macular degeneration. An ophthalmic composition as described herein wherein the age related macular degeneration is either dry or wet macular degeneration.

All the therapies described above utilize cyclodextrins solely as an excipient in pharmaceutical formulations containing other types of small molecule active ingredients for the treatment of AMD. None of the publications discussed suggest the use of cyclodextrins in therapeutically effective amounts as an active ingredient for the solubilisation of drusen and/or the treatment of dry AMD. Furthermore, measurement of effectiveness of those therapies often requires lengthy clinical trials to determine whether visual acuity has been improved.

What is needed is a therapy for dry AMD (a manifestation of decreased conductivity across a lipid laden retina-choroid interface) that targets the drusen that accumulate in the eyes of patients suffering from, or at risk for developing, dry AMD. Moreover, it is desirable to be able to discern quickly, i.e., within a matter of weeks or months, rather than years, whether such a therapy is effectively eliminating such drusen deposits, thereby decreasing the risk of vision loss due to dry AMD.

As used herein, the phrase “therapeutically effective amount” refers to concentrations cyclodextrins within the compositions of the invention that are effective for eliciting a therapeutic response in a patient, e.g., the solubilisation of drusen such that the damaging effects of its accumulation are avoided or ameliorated. Therapeutically effective amounts of cyclodextrin compounds for use in the invention are as described above. Therapeutically effective can also mean the frequency of dosing of the composition, as described above, or the duration of treatment, also as described above.

As used herein, the phrase “topical” means applied to the external surface of the eye, i.e. onto the cornea.

The treatment described herein is for long term use where in the composition is administered over an extended period of time. It is believed that the effects of the composition may only be seen after several days of treatment, if not weeks. Each cyclodextrin molecule that reaches the posteriori segment of the human eye is only able to excise it portion of waste material then the effects are linked to the concentration of cyclodextrin used in the composition, the frequency of dosing and the duration of treatment. We have found that we believe that approximately a 10% wt solution of a cyclodextrin is preferred. It is preferred that the composition is applied daily. Preferably the composition is applied once a day in the evening or in the morning. Optionally it may be applied twice a day—once in the morning and once in the evening. The treatment should be applied for several days, at least four weeks and preferably at least for 1, 2, 3, 4, 5, 6, 7, 8, 9, 10, 11, or 12 months. Optionally the treatment is stopped after the condition being treated has improved, as directed by the ophthalmologist, i.e. for a period of no more than 2 years, 20, 18, 16, or 14 months. When used as a preventative therapy or for maintenance dosing (a maintenance dose being a reduced concentration or a reduced frequency of dosing) then it may be used for an even longer duration of time such as for the duration of the patient's life so.

As used herein, the word “drusen” refers to one or more of the following definitions;
Hard Drusen: are small, distinct and far away from one another. This type of drusen may not cause vision problems for a long time, if at all.
Soft Drusen: “Soft” drusen are large and cluster closer together. Their edges are not as clearly defined as hard drusen. This soft type of drusen increases the risk for AMD
Reticular Pseudodrusen (aka Reticular Drusen; Subretinal Drusenoid Deposits; PseudoDrusen; Reticular Drusen): Although identified almost 30 years ago, reticular pseudodrusen (RPD) have been recently recognized as a distinctive phenotype. Unlike drusen, they are located in the subretinal space. RPD are strongly associated with late AMD, especially geographic atrophy, type 2 and 3 choroidal neovascularization, which, in turn, are less common in typical AMD. Precise composition of RPD is yet to be determined.
Cuticular Drusen (Basal Laminar Drusen): These are small (25-75 microns), yellow-white and nodular with OCT demonstrating these drusen to be blunted, triangular-shaped and below the RPE. FA strikingly reveals innumerable hyperfluorescent drusen that significantly outnumber the drusen noted clinically
Optic Nerve Drusen: Drusen can also occur in the optic nerve. These drusen are made up of protein and calcium salts and generally appear in both eyes. Unlike the drusen associated with AMD, optic nerve drusen (also known as optic disc drusen) are not related to aging and often appear in children. Optic nerve drusen usually do not affect vision, but some patients with these drusen may lose peripheral (side) vision.

Drusen can be visualised and their growth measured by the use of high density optical coherence tomography (OCT) macular scans that will identify the location of drusen, such as by using a Heidelberg Eye Explorer 2 (HEYEX2). Volumes of identified drusen and their geometry can be measured directly from these scans using suitable software, such as HEYEX2 platform software and also numerous software available to use on the MATLAB platform. The software can be used to measure changes in specific drusen of each patient over time both in terms of volume and shape.

In addition to modifying drusen the compositions described herein may also be used to treat Thickened Bruch's Membrane: Bruch's membrane calcifies and doubles in thickness between the ages of 10 and 90 years. There is a linear thickening due to deposits of collagen, lipids and debris. After the 30's its lipid concentration increases during life and consequently the fluid permeability and nutrient transport across the membrane decreases.

The compositions of the invention may be used alongside other active ingredients used for treating a number of eye diseases. That active ingredient may be present in the same composition as the cyclodextrin further include an additional active agent, such as a neuroprotective compound, an anti-angiogenic compound, or a neovascularization inhibiting compound. Anti-angiogenic compounds for use in the compositions and methods of the present invention may include anecortave acetate. Neovascularization inhibiting compounds for use in the compositions and methods of the present invention may include ranibizumab, bevacizumab, other VEGF inhibitors, and receptor tyrosine kinase inhibitors. Other compounds that could be useful in combination with the compositions of the present invention include anti-inflammatory agents (e.g., steroidal and non-steroidal agents); tyrosine kinase inhibitors; anti-infectives, (e.g., antibiotics, antivirals, and antifungals); antiallergic agents (e.g., antihistamines and mast cell stabilizers); cyclooxygenase inhibitors, (e.g., Cox I and Cox II inhibitors); combinations of anti-infective and anti-inflammatory agents; decongestants; anti-glaucoma agents, (e.g., adrenergics, β-adrenergic blocking agents, α-adrenergic agonists, parasympathomimetic agents, cholinesterase inhibitors, carbonic anhydrase inhibitors, and prostaglandin analogues, and combinations of such anti-glaucoma agents); antioxidants; nutritional supplements; drugs for the treatment of cystoid macular edema (e.g., non-steroidal anti-inflammatory agents); drugs for the treatment of ARMD, (e.g., angiogenesis inhibitors and nutritional supplements); drugs for the treatment of herpetic infections and CMV ocular infections; drugs for the treatment of proliferative vitreoretinopathy (e.g., antimetabolites and fibrinolytics); wound modulating agents (e.g., growth factors); antimetabolites; and neuroprotective drugs (e.g., eliprodil). It is further contemplated that the compositions of the present invention may be used in conjunction with photodynamic therapy.

Cyclodextrins are known to bind lipids in ocular tissue. As well as effects upon the retina this lipid binding may occur in other parts of the eye and adnexa where abnormal lipid is known to accumulate. These areas include:

1. The eyelid—for example in removing or preventing the formation of Xanthelasma
2. The removal of fatty deposits commonly found subconjunctivally and/or episclerally
3. The cornea—for example with helping to remove the arcus juvenilis or age related arcus.

Additionally in helping with corneal trauma, infections and inflammation.

Topically applied cyclodextrin may reduce the lipid amount in these areas/structures listed above and provide the above listed benefits in addition to their other effects as described herein.

The compositions of the invention may include an additional active agent, as described herein, it is not preferred due to the ability of the cyclodextrin to easily form clathrates with the active ingredient and affect the ability of the cyclodextrin to perform its primary role in the compositions of this invention. It is contemplated though that the compositions of the invention may be administered in conjunction with additional active agents for treating retinal disorders where the additional active agents are administered in separate compositions either concurrently with administration of the compositions of the invention, prior to their administration or after their administration. For example, the compositions of the invention could be administered minutes, hours, days, or weeks prior to or after administration with an additional active agent for treating retinal disorders. In preferred embodiments, the compositions of the invention will be administered with the additional active agent during the same office visit. Alternatively, the compositions of the invention could be administered to a patient from one day to a month or two months prior to administration of the additional active agent.

When used in conjunction with additional active agents for the treatment of AMD or other retinal disorders, the compounds of the invention function to physiochemically solubilize the drusen present in the patient's eye, shrinking the size of such drusen, or eliminating them altogether, within a matter of days, weeks or months. The use of an additional active agent can then provide longer lasting neuroprotection and/or inhibition of angiogenesis or neovascularization, thereby stabilizing or improving the patient's vision. In some aspects of the invention, the therapeutically effective amount of the cyclodextrin and the additional active agent are present in the same composition. In this case, a low concentration of a cyclodextrin may be incorporated in the composition to solubilize or act as a penetration-enhancer for the additional active agent. The additional active agent may be incorporated as an inclusion complex with the cyclodextrin, which will help carry the additional active agent to the active site. In such compositions, a separate, therapeutically effective amount of at least one cyclodextrin is present in the composition. The therapeutically effective amount of the cyclodextrin is not part of an inclusion complex. The cyclodextrin that is present in a therapeutically effective amount may be the same cyclodextrin, or a different cyclodextrin, from the cyclodextrin that is acting to solubilize the additional active agent.

Preferably the cyclodextrin is in solution in the composition.

### Examples

### Formulation

A formulation was prepared by dissolving 2 kg of (2-hydroxypropyl)-β-cyclodextrin in 201 of water for injection in a sterile environment. Novelia® ophthalmic multi-dose bottles from Nemera were filed with 10 ml of the composition.

The resulting formulation has the following composition

10% wt of (2-hydroxypropyl)-β-cyclodextrin
qs water.

Formulation 1

| component name | % w/v | type |
|---|---|---|
| Hydroxypropyl-beta-cyclodextrin | 10.000 | Active |
| Na₂HPO₄ | 0.374 | Buffer |
| Na₂HPO₄ | 0.460 | Buffer |
| Water for injection | qs to 100 mL | Diluent |

Formulation 2

An osmolality of 290 mOsm/Kg.

| component name | % w/v | type |
|---|---|---|
| Hydroxypropyl-beta-cyclodextrin | 10.000 | Active |
| Na₂HPO₄ | 0.474 | Buffer |
| Na₂HPO₄ | 0.560 | Buffer |
| Water for injection | qs to 100 mL | Diluent |

Efficacy Data

The formulation 2 as described above is administered to a patient suffering from intermediate age related macular degeneration topically by the patient at least twice a day for 6 months

Before the study the patient is assessed to determine the extent of hard and soft drusen in the eye and the size using an optical coherence tomography (OCT) and colour fundus photographs.

The patient is re-assessed monthly using the OCT and at 6 months with colour fundus photograph as well.

We have found that the drusen sized>125 um reduced in number and size indicating that the cyclodextrin is reaching the drusen and causing there to be shrinkage.

### Clinical Investigation Plan: Overview

### 1 Study Population

Adult subjects over 50 years of age. Subjects will be screened for eligibility at the screening/baseline visit. Fifteen participants of either sex are included in the study.

### 1.1 Inclusion Criteria

### A Subject Included in the Study Must:

be over 50 years of age.
Have a life expectancy of more than 6 months' (the length of the study).
Have been informed of, and be able to perform, the study treatments and procedures and have signed an informed consent form.
Have provided authorisation to use and disclose information for research purposes.

### 1.2 Exclusion Criteria

### A Subject Will not be Included in the Study if he or she:

Is a current smoker.
Is known to suffer from an allergy or hypersensitivity to any of the components within the medical device.
Is taking any other eye drops containing cyclodextrin.
Is or has used contact lenses within the last 4-7 days
Has a history of inflammatory corneal ulcer or uveitis within the last 12 months'
Has allergic rhinitis that is current or susceptible to reactivation during the study
Has had cataract or corneal surgery in the last 12 months.
Is currently participating in any other clinical trial, or has participated in another clinical trial within 3 months prior to the screening/baseline visit.
Is not physically able to perform study procedures
Has a history of drug/alcohol abuse, mental dysfunction or other factors limiting their ability to cooperate fully.
Has any other condition, which in the opinion of the investigator, would make the subject not a suitable candidate for the study.

### 2 Study Procedures

### 2.1 Trial Design

Patients are provided with a 10 ml Novelia bottle containing hydroxypropyl-beta-cyclodextrin in a 10% solution with water, as disclosed above Formulation 2. The participants apply the eye drops a minimum of twice every day or as or when needed to relieve dry eye symptoms during the trial period.

### 2.2 Summary of Assessment Timeline

At the baseline/screening visit informed consent, eligibility and medical history is taken for each participant. Concomitant medications are recorded at the baseline visit and any subsequent changes to the medication are recorded at each visit. An adverse event form will be recorded at each visit.

A patient questionnaire will be completed at each visit/discussion. At the baseline screening, the patient questionnaire will be taken home with the patient in preparation for the 2-week phone call.

A full ocular examination plus retinal OCT scan is performed at each visit to the treatment centre (monthly).

Schirmer's test and tear-film break-up time will be evaluated after 1, 3 and 6 months to test the effect of the formulation on tear fluid production.

This can also be found summarised in the table below.

| | Screening/ | 2 | 1 | 2 | 3 | 4 | 5 | 6 | |
|---|---|---|---|---|---|---|---|---|---|
| | baseline | weeks | month | months | months | months | months | months | Withdrawal |
| Informed | X | | | | | | | | |
| consent | | | | | | | | | |
| Eligibility form | X | | | | | | | | |
| Medical history | X | | | | | | | | |
| Concomitant | X | X | X | X | X | X | X | X | X |
| medication | | | | | | | | | |
| Patient | X | X | X | X | X | X | X | X | |
| questionnaire | | | | | | | | | |
| Full ocular | X | | X | X | X | X | X | X | X |
| examination | | | | | | | | | |
| including OCT | | | | | | | | | |
| Schirmer's test | X | | X | | X | | | X | X |
| and TFBUT | | | | | | | | | |
| AE form | | X | X | X | X | X | X | X | X |
| Withdrawal | | | | | | | | | X |
| form | | | | | | | | | |

### 2. Trial Intervention

### 2.3 Description of the Device

The Novelia device is manufactured by the Nemera and is CE marked as a class IIa medical device. The Novelia device has also been approved as a container closure system for medicinal products Eysano (timolol), Eydelto (Dorzolamide), Eylamdo (Dorzolamide/Timolol) and Eykappo (chloramphenicol).

### 2.4 Dosing Regimen

Patients apply the drops at least twice a day (morning and night) and as required. Patients should keep a note of how often they have applied the drops per day.

### 2.5 Subject Compliance

Patients provided with a form to fill in each day, which lists the number of drops that they have administered. At the two-week phone call from the investigator to the patient, the investigator will determine if the patient is complying with the instructions for use and/or has any issues relating to administration of the device.

### 2.6 Concomitant Therapy

All concomitant medication and current and past therapies in the last 12 months are recorded at the screening visit and any change in the concomitant medications will be recorded at each visit.

### 3 Subject Completion and Withdrawal

### 3.1 Withdrawal of Subjects

Visit windows of +/−10 days should ensure visit attendance; non-attendance for visits will prompt follow-up by telephone. However, a delayed visit should be entered in the database. An appointment is only defined as missed if the delayed visit is within 10 days of the next pre-defined trial visit date. OCT

### 3.2 Drusen Analysis

Drusen growth/shrinkage and change in frequency is measured by the use of high density optical coherence tomography (OCT) macular scans that will identify the location of drusen, such as by using a Heidelberg Eye Explorer 2 (HEYEX2). Volumes of identified drusen and their geometry can be measured directly from these scans using suitable software, such as HEYEX2 platform software and also numerous software available to use on the MATLAB platform. The software can be used to measure changes in specific drusen of each patient over time both in terms of volume and shape.

### BRIEF DESCRIPTION OF THE DRAWINGS

FIGS. 1A-1C show an en face OCT patient scan with more frequent and larger drusen a) colour fundus photograph b) fluorescein angiography c) cross sectional scan of the retinal layers.

FIGS. 2A-C show an en face OCT patient scan with less frequent and smaller drusen a) colour fundus photograph b) fluorescein angiography c) cross sectional scan of the retinal layers.

### DETAILED DESCRIPTION OF THE DRAWINGS

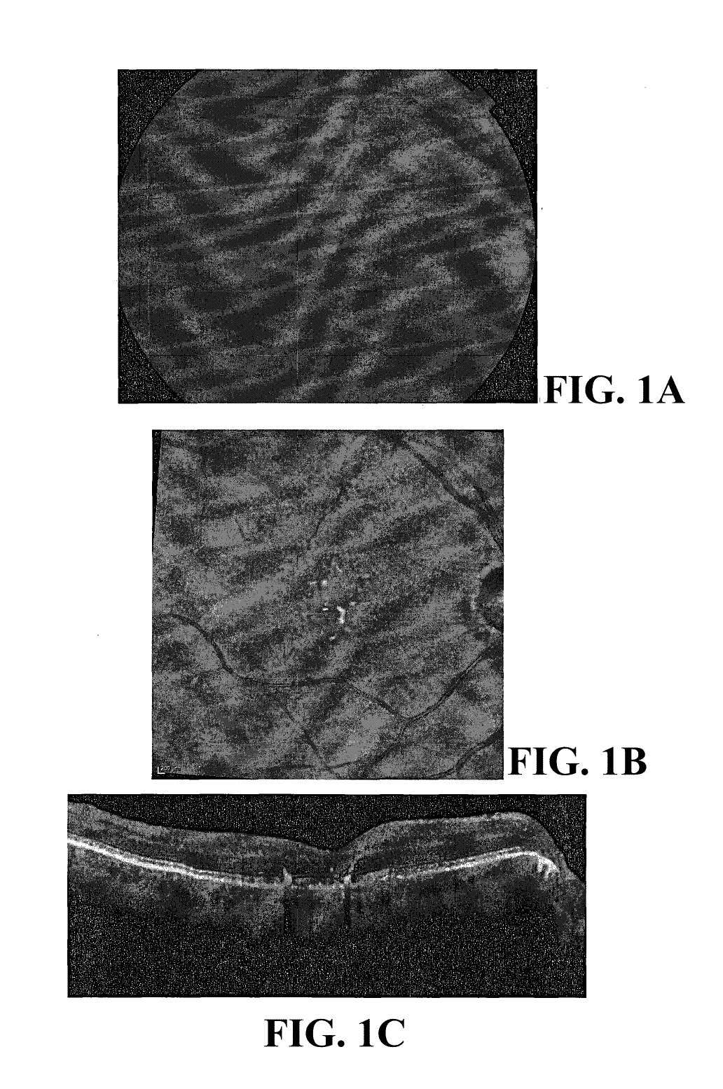

FIGS. 1A-C show an en face OCT patient scan with more frequent and larger drusen a) colour fundus photograph b) fluorescein angiography c) cross sectional scan of the retinal layers.

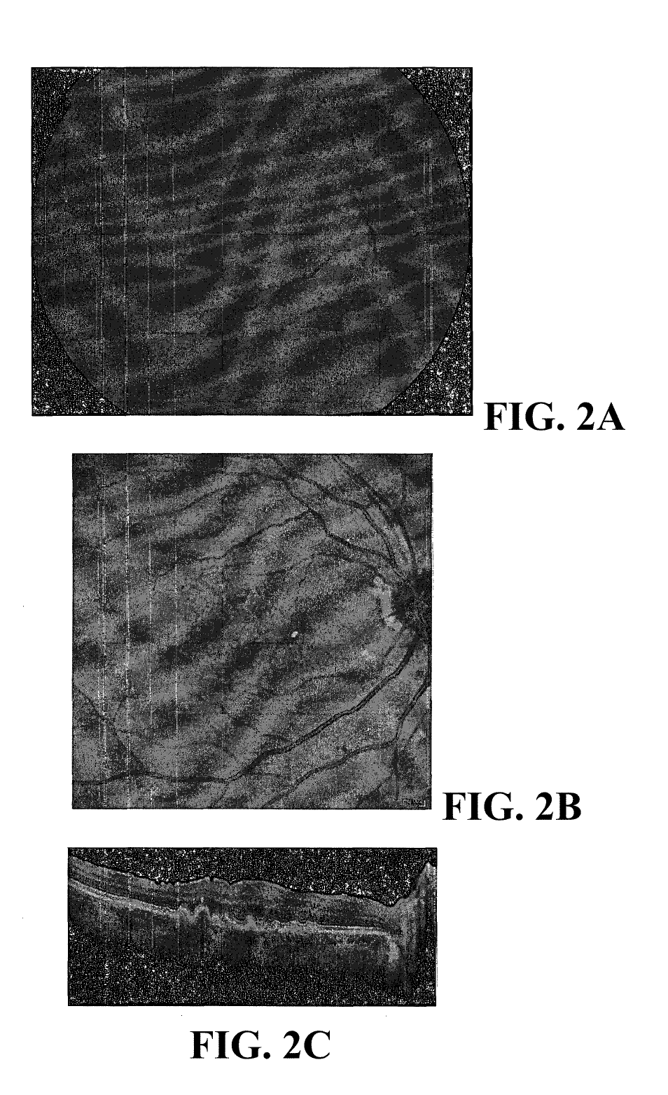

FIGS. 2A-C show an en face OCT patient scan with less frequent and smaller drusen a) colour fundus photograph b) fluorescein angiography c) cross sectional scan of the retinal layers.

1. An ophthalmic topical composition comprising a cyclodextrin as the sole active ingredient and any pharmaceutically acceptable excipient therefor for use in the removal of drusen, or reduction in the size or the number of drusen, or the prevention of the formation of drusen, in the eye of a human.

2. An ophthalmic topical composition as claimed in claim 1 wherein, the concentration of cyclodextrin solution is less than 22% wt. in an aqueous vehicle.

3. An ophthalmic composition as claimed in claim 2 where, in the remaining part of the composition that is not cyclodextrin or water is selected from one of more of the following excipients:
a) tonicity agent,
b) wetting agent,
c) viscosity agent, and
d) preservative.

4. An ophthalmic composition as claimed in claim 1 wherein, the composition comprises a component that acts as an artificial tear and/or an eye lubricant.

5. An ophthalmic topical composition as claimed in claim 1 wherein, the composition is for use in the removal of drusen in the eye of a human patient suffering from age related macular degeneration.

6. An ophthalmic topical composition as claimed in claim 5 for removal of drusen within, or between the retinal pigment epithelium and Bruch's membrane of the eye.

7. An ophthalmic topical composition as claimed in claim 5 for removal of drusen, and/or reduction in the size or number of drusen, in the macula.

8. An ophthalmic topical composition as claimed in claim 1 wherein, the composition is for use in the prevention of the formation of drusen in the eye of a human eye.

9. An ophthalmic topical composition as claimed in claim 1 wherein the patient has previously been identified as being susceptible to the development of macular degeneration.

10. An ophthalmic composition as claimed in claim 1 for preventing acute macular degeneration.

11. An ophthalmic composition as claimed in claim 1 for treating macular degeneration.

12. An ophthalmic composition as claimed in claim 1, wherein the macular degeneration is either dry or wet macular degeneration.

13. An ophthalmic composition as claimed in claim 1 wherein, the macular degeneration is wet macular degeneration.
